(19) United States
(12) Patent Application Publication
(10) Pub. No.: US 2002/0040051 A1
(43) Pub. Date: Apr. 4, 2002

(54) Solid dispersion of ipriflavone for oral administration and its manufacturing methods

(76) Inventors: Hai-Bang Lee, Daejeon (KR); Gil-Son Khang, Daejeon (KR); Je-Kyo Jeong, Daejeon (KR); Jeung Ku, Kwanyang-shi (KR); Sun-Hang Cho, Daejeon (KR)

Correspondence Address: LOWE HAUPTMAN GOPSTEIN, GILMAN AND BERNER LLP, SUITE 310, 1700 DIAGONAL ROAD, ALEXANDRIA, VA, 22314

(21) Appl. No.: 09/843,146

(22) Filed: Apr. 27, 2001

(30) Foreign Application Priority Data
Aug. 18, 2000 (KR) 2000-47918

(51) Int. Cl. A61K 9/48; A61K 9/20; A61K 31/352

(52) U.S. Cl. 514/456; 424/452; 424/465

(57) ABSTRACT

This invention relates to an ipriflavone-containing pharmaceutical agent for oral administration with improved bioavailability, wherein ipriflavone is solid-dispersed in the presence of a water-soluble polymer, an absorption fortifier, and an excipient while the crystal of said pharmaceutical agent is prepared in an amorphous form at the same time so that said ipriflavone can be enclosed in said water soluble polymer, and thus even a little amount as well as lower number of dosage of said ipriflavone pharmaceutical agent can increase the effective blood concentration of said ipriflavone pharmaceutical agent and the solubility for the body fluid in the gastrointestinal tract, thereby remarkably improving the bioavailability of said agent which can much reduce both the uncomfortableness in its usage and the burden that is usually laid on the gastrointestinal tract by a heavy dose and also increase the stability during a long-term storage.

### BACKGROUND OF THE INVENTION

1. Field of the Invention

This invention relates to an ipriflavone-containing pharmaceutical agent for oral administration with improved bioavailability, and more particularly, to an ipriflavone-containing pharmaceutical agent for oral administration wherein ipriflavone, commonly used as a therapeutic agent for osteoporosis, is solid-dispersed in the presence of a water-soluble polymer, an absorption fortifier, and an excipient while the crystal of said pharmaceutical agent is prepared in an amorphous form at the same time so that said ipriflavone can be enclosed in said water soluble polymer, and thus even a little amount as well as lower number of dosage of said ipriflavone pharmaceutical agent, as compared to those of conventional medicational therapies, can increase the effective blood concentration of said ipriflavone pharmaceutical agent and the solubility for the body fluid in the gastrointestinal tract, thereby remarkably improving the bioavailability of said agent which can much reduce both the uncomfortableness in its usage and the burden that is usually laid on the gastrointestinal tract by a heavy dosage and also increase the stability during a long-term storage.

2. Description of the Related Art

Osteoporosis is a disease resulted from various reasons such as dietary failure, famine, senility, neurologic as well as endocrinal reasons and its symptom is well characterized by having an enlarged airbone fossa due to thinned compact bones which is triggered by the decrease in calcine in bone structure without any external abnormalities (Donga Color World Encyclopedia, Vol. 3, p. 353, 2000).

One of the therapeutic agents widely used in treating and preventing the osteoporosis is ipriflavone (3-phenyl-7-isopropoxy-4H-1-benzopyrane-4-one). A line of study on the pharmacological mechanism of the ipriflavone discloses that ipriflavone can improve the decrease in bone mass via direct inhibition of bone resorption together with the inhibition of bone resorption due to the increased secretion of calcitonin by estrogen [U. Lerner and B. B. Fredholem. Biochem. Pharm. 34, 937 (1985); Anon. Phase III, Drug Profiles, 4, 10 (1994)]. However, ipriflavone is a highly water-insoluble substance having a water solubility of 1 μg/mL and with a high crystallinity thus necessitating orally administer a large amount of ipriflavone for a relatively long period of time in order for the ipriflavone to reach the minimum effective blood concentration sufficient for the effective expression of its desired pharmaceutical action [T. Sato et al., Endocrinol., Jpn., Vol. 33, p. 23 (1986); I. Yamazaki et al., Life Sci., Vol. 38, p.951 (1986)]. Since the general preparation form of ipriflavone is crystalline powder and its solubility to body fluids is near zero, ipriflavone is freeze-crushed for the purpose of increasing the surface area, mixed with various excipients, and finally prepared into capsules or tablets to be in market. However, this method is again not very efficient in bioavailabilty.

Ipriflavone, based on clinical data, has been administered 3-4 times daily with 200 mg per each administration, i.e., 600-800 mg of daily administration based on a healthy adult male (18-60 yr), and the blood concentration of ipriflavone can reach an effective level on the 3^{rd} or 4^{th} day of the administration. Therefore, the necessity of a heavy dose as well as a lengthy duration of administration of ipriflavone to exhibit its therapeutic effect becomes a nuisance for a patient and it usually needs to take digestive agents as an aid to lessen the burden usually imparted on the gastrointestinal tract due to the nature of the long-term medication.

Although ipriflavone has an excellent pharmacological effect, it has a low bioavailability (i.e., a low body absorption rate) and this is because the solubility of crystallized ipriflavone agent is closely associated with the absorption in gastrointestinal tract. That is, the dissolution rate in gastrointestinal tract becomes the important step of determining the rate of body absorption and thus the lower the dissolution rate the slower the absorption rate in the gastrointestinal tract. Therefore, it becomes quite obvious that the dissolution of a drug from the digestive juices in the gastrointestinal tract as well as the diffusion in the absorption interface in intestine should be rapidly proceeded (Kelm et al., U.S. Pat. No. 5,281,420).

U.S. Pat. No. 5,504,105 discloses a method to remedy the drawback of ipriflavone for being water-insoluble by oral administation of ipriflavone in the form of a mixture comprising soya lecithin, medium-chain glycerides, white chocolate and hydrogenated vegetable oil. However, this method is mainly concerned about the improvement of bioavailability and the practical rate of bioavailability was increased only by a factor of 1.5 thus becoming inefficient and this method was also shown to be economically limited.

Another method of resolving the problem of water-insolubility of ipriflavone was recently disclosed and this method employs a freeze-crusher to crush crystallized ipriflavone thus resulting in the increase in the surface area of an ipriflavone-containing pharmaceutical agent thereby increasing its solubility [Vervaet et al., Pharm. Res., Vol. 14, p.1644 (1997)]. However, this method is also very restricted in its use because air bubbles will become attached to the surface of said pharmaceutical agent as the crystallized ipriflavone is crushed to reach a certain size and they are hard to get rid of and the difficulty of disintegration of powder will further decrease the solubility.

Korea Patent Application. No. 96-21056 discloses a method to increase the rate of absorbing ipriflavone by micro-emulsifying ipriflavone via oil components and surfactants. However, this method is not also preferred because the surfactants used in this method is known to be toxic to the gastrointestinal tract.

Korea Patent Application No. 96-5136 discloses a method wherein ipriflavone is dissolved in ethanol or isopropanol or acetone, and said mixture solution is then mixed again with sufficient amount of water to be prepared in a hydrosol state, and finally prepared in the form of an injection or a tablet. However, this method is also not recommended because the process according to this method is very complex and also not economical due to relatively high cost of unit production.

Korea Patent Application No. 96-33693, also invented by the present inventors, discloses a method wherein ipriflavone is solid-dispersed in a water-soluble polymer and an excipient, during which the size of crystallized ipriflavone is diffused to 10 nm-20 μm thus increasing approximately 10-15 times the area under the curve for the blood concentration. The advantages of this method are that the pharmaceutical preparations can be easily prepared and the rate of absorption in body for these pharmaceutical preparations can be increased approximately 10-15 times. However, this method is also not advantageous in that it cannot provide a good long-term storage stability and thus the amorphous property of ipriflavone can be converted to a crystalline form during storage and thus decreasing the rate of absorption in body.

Consequently, the ipriflavone used as an active ingredient in preparing conventional pharmaceutical agents is highly water-insoluble and also has a high crystallinity, therefore, it is highly required to develop a new method that can resolve the drawbacks.

### SUMMARY OF THE INVENTION

Therefore, the inventors of the present invention further developed the above method disclosed in their Korea Patent Application No. 96-33693 containing ipriflavone wherein ipriflavone is solid-dispersed in the presence of a water-soluble polymer and an excipient while during which the crystalline size of said pharmaceutical agent is diffused to 10 nm-20 μm by adding an absorption fortifier during the preparation process thus improving the rate of drug absorption and also inhibiting the recrystallization of ipriflavone.

Therefore, the object of the present invention is to improve the rate of ipriflavone absorption in the body by increasing the solubility by using a vehicle of a water-soluble polymer which is easily dissolved in the body and said water-soluble polymer containing ipriflavone can be delivered to the gastrointestinal tract along with an added absorption fortifier, and to provide an ipriflavone-containing pharmaceutical agent for oral administration having improved bioavailability as well as an excellent stability during a long-term storage by reducing both the uncomfortableness in its usage and the burden that can be laid on a gastrointestinal tract by a heavy dose.

### DETAILED DESCRIPTION OF THE INVENTION

This invention relates to an ipriflavone-containing pharmaceutical agent for oral administration with improved bioavailability wherein 1.0 part by wt of ipriflavone is solid-dispersed in the presence of 0.1-10 parts by wt of a water-soluble polymer, during which is also solid-dispersed simultaneously 0.01-5 parts by wt of an absorption fortifier.

This invention is explained in more detail as set forth hereunder.

This invention relates to an ipriflavone-containing pharmaceutical agent, wherein even a little amount of administration and lower number of dosage of said ipriflavone-containing pharmaceutical agent, as compared to those of the conventional medications, can improve the bioavailability because the water-insoluble and crystalline ipriflavone can be solid-dispersed by a water-soluble polymer and the crystallinity of ipriflavone thus reducing both the nuisance in its usage due to a frequent as well as a heavy dose and the burden that can be laid on the gastrointestinal tract by a heavy dose.

One of the features of the present invention is characterized by using a water-soluble polymer, a highly soluble in the gastrointestinal tract, as an agent to solid-disperse ipriflavone.

Another feature of the present invention is that 1.0 part by wt of ipriflavone can be solid-dispersed in the presence of 0.1-10 parts by wt of a water-soluble polymer although there can be a variation in the solid-dispersion depending on the property of a given water-soluble polymer.

Here, the water soluble polymer used in the present invention is one or a mixture of more than two selected from the group consisting of polyvinylpyrrolidone, polyvinyl alcohol, polyethylene glycol, gum Arabic, dextran, dextrin, gelatin, methyl cellulose, ethyl cellulose, hydroxyethyl cellulose, hydroxypropyl cellulose, carboxymethyl cellulose, Poloxamer, Fluronic, and Polysorbate.

The examples of an absorption fortifier are one or a mixture of more than two selected from the group consisting of citric acid, alginic acid, ascorbic acid, bile acid, lithocolic acid, cholic acid, deoxycholic acid 5β-cholanic acid, trihydroxy cholane, cholesterol, cholesteryl oleate, cholesteryl oleate, cholesteryl palmitate, cholesteryl acetate, cholesteryl stearate, salicylic acid, mannitol, xylitol, dextrose, glucose, sucrose, galactose, sorbitol, lactose, fructose, maltose, pentaerithritol and pentaerithritol tetraacetate.

A disintegrator used in the present invention can be selected from the group consisting of hydroxypropyl starch, sodiumstarch-glycolate, magnesium stearate and calcium stearate.

The present invention also employs a lubricant during the preparation of ipriflavone-containing pharmaceutical agent and said lubricant can be selected from the group consisting of urea and dextrose monohydrate, and the selected lubricant is recommended to use 0-20% by wt with respect to the 100% by wt of the ipriflavone-containing pharmaceutical agent.

Still another feature of the present invention lies in that the ipriflavone-containing pharmaceutical agent can be prepared by randomizing crystalline size of ipriflavone when ipriflavone is solid-dispersed in the presence of a water-soluble polymer and the recrystallization of said randomized ipriflavone can be also prevented. The randomization of crystalline size of ipriflavone can be adjusted based on the manufacturing temperature, stirring speed, molecular weight of water-soluble polymer, concentration of manufacturing and the concentration of an added excipient.

A general solid dispersion method is used when solid-dispersing said ipriflavone in the presence of a water-soluble polymer and the examples of the general solid dispersion methods include solvent process, melt extrusion, fusion process, mixed-grinding technology, and thermal-mechanochemical process, a method wherein heat is applied to the above methods.

The above-mentioned methods can adjust the crystalline size of the manufactured ipriflavone-containing pharmaceutical agent and each method has its own merits and demerits with respect to cost and process.

The present invention employs a conventional solvent process and a melt extract method for solid-dispersion and final products are manufactured in the form of powdered granules and pellets generated upon extrusion, i.e., hard and soft capsules, tablets and pills.

The method of using solvents to manufacture an ipriflavone-containing pharmaceutical agent according to the present invention is explained hereunder.

In the present invention, ipriflavone is dissolved in a good solvent such as acetone, dichloro methane, ethanol, and the mixture of these, further added along with a water-soluble polymer, and then manufactured into solid-dispersed white powders by air-dry using a fluidized spray dryer, which is well-known to a person in this art.

The method of manufacturing an ipriflavone-containing pharmaceutical agent by means of a mixed method of melt-extrusion is as follows.

In the present invention, the ipriflavone-containing pharmaceutical agent is prepared by mixing the ipriflavone with a water-soluble polymer, such as polyvinylpyrrolidone and polyethylene oxide, and an agent which serves as an inhibitor of recrystallization as well as an absorption fortifier such as citric acid; adding said mixture into an injection extruder via simple fusion; and solid-dispersing by adjusting the temperature in said extruder. Here, a little amount of urea or magnesium stearate is added as a lubricant into the mixture of ipriflavone and a water-soluble polymer to provide a necessary lubrication during the manufacturing process. Further, a mold is installed on the die at the end of said injection extruder in the form of a thread and is cut out to have the same length as the diameter of said mold and this enables the final pharmaceutical preparations be made in the form of either capsules or powder. The ipriflavone that goes through with the above-mentioned method will be converted into an amorphous form and thus it can be more easily dissolved by the body fluid in the periphery of the wall of the gastrointestinal tract and the diffusion can be more easily facilitated thereby increasing the absorption of ipriflavone in human body.

An excipient that can be used in the present invention can be selected from the group consisting of crystalline cellulose, corn starch, D-mannitol, and lactose.

In manufacturing an ipriflavone-containing pharmaceutical agent according to the present invention, ipriflavone is not only solid-dispersed and converted into an amorphous form but said amorphous ipriflavone is also prevented from being recrystallized thus contributing to the increase in the rate of ipriflavone absorption in the body. This will then reduce the amount of daily dosage that osteoporosis patients need to take according to the traditional therapies thus alleviating the nuisance resulted from frequent and heavy medications.

This invention is further illustrated by the following examples. However, these examples should not be construed as limiting the scope of this invention in any manner.

### EXAMPLE 1

First, 90 g of ipriflavone (Hongsung Chem. Co., Ltd.) and 20 g of citric acid were evenly dissolved in 1,400 g of acetone. Then, 90 g of polyvinylpyrrolidone (Hongsung Chem. Co., Ltd.), a water-soluble polymer, was evenly dissolved in 200 g of a mixed solvent of acetone/ethanol (1:1, v/v). Said ipriflavone-containing solution and said polyvinylpyrrolidone solution were added into a fluidized-bed spray-dryer (Model, Uinglatt, Glatte Co., Germany), sprayed so that ipriflavone can be distributed in polyvinylpyrrolidone, and then placed under air-dry to produce an ipriflavone-containing pharmaceutical agent in the form of amorphous white powder for use in animal experimentations. Here, the ratio of parts by wt among ipriflavone, polyvinylpyrrolidone, and citric acid was adjusted to be 45:45:10. The conditions during the spray were 50° C. for the temperature of air influx, 30 psi of air pressure for spray, and 12 mL/min for the spray speed.

### EXAMPLE 2

First, 20 g of ipriflavone (Hongsung Chem. Co., Ltd.) and 2 g of of alginic acid were evenly mixed with 20 g of polyethylene glycol (MW: 32,000 g/mole), solid-dispersed by placing under simple fusion at 90° C. using a hot plate, and freeze-crushed in a liquid nitrogen to be prepared as a white powdered ipriflavone-containing pharmaceutical agent having a size of 10 μm to be used in animal experiments.

### COMPARATIVE EXAMPLE 1

Ipriflavone (Hongsung Chem. Co., Ltd.) was directly used without adding any other substances or treatments.

### COMPARATIVE EXAMPLE 1

Ipriflavone-containing pharmaceutical agent was prepared the same as in the example 1 with the exception that citric acid was not added. Experimental Example 1: Animal Experiment and Analysis of Collected Blood

(1) Experimental Animal

Animals used in this experiment were male Sprague-Dawley (SD) mice which were kindly supplied by Daehan Biolink Co., Ltd. in Korea. The body weight of the mice used in the experiment ranged from 240 to 260 g. The mice were allowed to adapt to a new laboratory for more than a week before use and each control group consisted of more than three mice.

(2) Medication

All the experimental mice in examples 1 and 2, and comparative examples 1 and 2 were forced to fast for 24 hr before the experiment and then orally administered with 50 mg of ipriflavone after suspending it in deionized water per 1 kg of body weight.

(3) Blood Collection and Analyses

The concentration of ipriflavone in blood plasma absorbed through the gastrointestinal tract of each mouse after oral administration with an ipriflavone-containing pharmaceutical agent was measured as follows.

Blood samples of the experimental SD mice were collected at certain intervals by means of a polyethylene catheter which was already inserted into the carotid of SD mice by using a heparin-treated syringe. The amount of blood sample collected from each SD muse was 150 μL per each collection and was replenished with 150 μL of heparin immediately after the blood collection. The collected blood samples were then centrifuged for 5 min to separate blood plasma and stored said blood plasma at −20° C. until they were needed for the analyses of the concentration. The protein in said blood plasma was agglutinated by adding 2.5 times in excess the amount of acetonitrile with respect to the volume of blood plasma, mixed for 30 sec using an agitator, centrifuged for 5 min at 5,000 rpm and then the ipriflavone in the blood plasma was assayed via HPLC. The conditions used in HPLC column was as follows: the column was Lichrosorb RP-18 (10 μm, 3.9×300 mm, phase separations, USA); the mobile phase was a mixture at pH 3 with the volumetric ratio of acetic acid buffer solution/acetonitrile/methanol being 40:35:25 (v/v%); the flow rate was 2.0 mL/min; detecting UV wavelength was 254 nm; and the amount of influx was 50 μL.

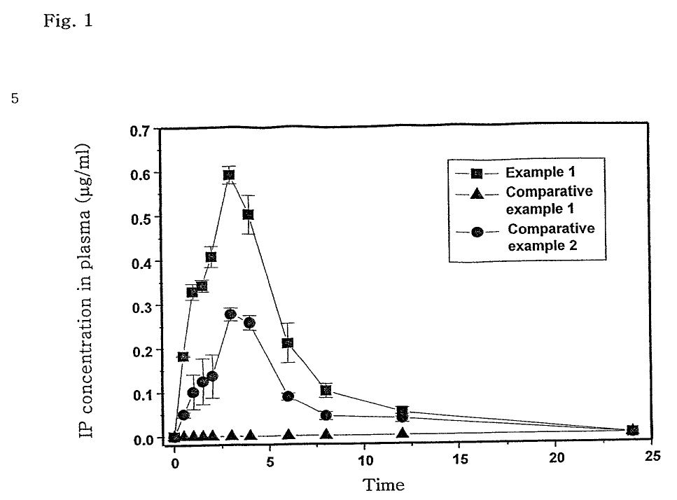

The change of concentration of the ipriflavone-containing pharmaceutical agent in blood in the example 1 and comparative examples 1 and 2 are shown in the FIG. 1 and the pharmacokinetic parameters are shown in the following table 1.

TABLE 1

| Pharmacokinetic | | Comparative | Comparative |
|---|---|---|---|
| Parameters | Example 1 | Example 1 | Example 2 |
| Absorption Rate | 0.427 ± 0.057 | Unable to | 0.320 ± 0.048 |
| Coefficient (hr⁻¹) | | measure | |
| Removal Rate | 0.353 ± 0.052 | Unable to | 0.278 ± 0.027 |
| Coefficient (hr⁻¹) | | measure | |
| Half-life of | 1.750 ± 0.192 | Unable to | 1.501 ± 0.175 |
| Absorption (hr) | | measure | |
| Half-life of | 2.252 ± 0.333 | Unable to | 2.001 ± 0.327 |
| Removal (hr) | | measure | |
| Time of Retention | 0.520 ± 0.147 | Unable to | 0.470 ± 0.154 |
| (hr) | | measure | |
| Time to reach | 3.000 ± 0.000 | Unable to | 3.000 ± 0.000 |
| Highest Blood | | measure | |
| Concentration (hr) | | | |
| Highest Blood | 0.591 ± 0.021 | Unable to | 0.276 ± 0.015 |
| Concentration | | measure | |
| (μg/ mL) | | | |
| Blood | 3.214 ± 0.201 | Unable to | 1.517 ± 0.207 |
| Concentration | | measure | |
| Area under the | | | |
| curve (μg × | | | |
| hr/mL) | | | |

N.B. The above values shown in Example 1 and Comparative Example 2 represent “average ± standard deviation”.

According to the table 1 and FIG. 1, the bioavailability of the ipriflavone-containing pharmaceutical agent in example 1 was much greater than when the ipriflavone was used alone without the addition of any other additives and the bioavailability almost doubled when compared to that in comparative example 2, thus implying that the ipriflavone-containing pharmaceutical agent according to the present invention is quite effective in preventing and treating osteoporosis.

As mentioned above, the ipriflavone-containing pharmaceutical agent according to the present invention is shown to have much improved bioavailability. Therefore, even with a relatively lesser amount and a lesser number of administration, as compared to the conventional methods, said agent can be facilitated to reach the effective blood concentration, can avoid gastric indigestion that is commonly present in most osteoporosis patients under the conventional medications due to relatively heavy and frequent doses, can provide a much easier and more economical manufacturing method, and can be applied to other water-insoluble crystalline pharmaceutical agents that require sustained release.

What is claimed is:

1. An ipriflavone-containing pharmaceutical agent with improved bioavailability for oral administration, wherein 1.0 part by wt of ipriflavone is solid-dispersed in the presence of 0.1-10 parts by wt of a water soluble polymer and 0.01-5 parts by wt of an absorption fortifier, and prepared in an amorphous pharmaceutical agent.

2. The ipriflavone-containing pharmaceutical agent with improved bioavailability for oral administration according to claim 1, wherein said water soluble polymer is one or a mixture of more than two selected from the group consisting of polyvinylpyrrolidone, polyvinyl alcohol, polyethylene glycol, gum Arabic, dextran, dextrin, gelatin, methyl cellulose, ethyl cellulose, hydroxyethyl cellulose, hydroxypropyl cellulose, carboxymethyl cellulose, poloxamer, pluronic and polysorbate.

3. The ipriflavone-containing pharmaceutical agent with improved bioavailability for oral administration according to claim 1, wherein said absorption fortifier is one or a mixture of more than two selected from the group consisting of citric acid, alginic acid, ascorbic acid, bile acid, lithocolic acid, cholic acid, deoxycholic acid 5β-cholanic acid, trihydroxy cholane, cholesterol, cholesteryl oleate, cholesteryl oleate, cholesteryl palmitate, cholesteryl acetate, cholesteryl stearate, salicylic acid, mannitol, xylitol, dextrose, glucose, sucrose, galactose, sorbitol, lactose, fructose, maltose, pentaerithritol and pentaerithritol tetraacetate.

4. The ipriflavone-containing pharmaceutical agent with improved bioavailability for oral administration according to claim 1, wherein said ipriflavone-containing pharmaceutical agent can be prepared as powdered granules, capsules, tablets and pills.
